(19) United States
(12) Patent Application Publication
(10) Pub. No.: US 2026/0345816 A1
(43) Pub. Date: Nov. 5, 2026

(54) NEW EPITHELIAL PERMEATION ENHANCER

(71) Applicant: ROQUETTE FRERES, LESTREM (FR)

(72) Inventor: Damien TRUFFIN, LESTREM (FR)

(21) Appl. No.: 19/488,304

(22) PCT Filed: May 28, 2024
(86) PCT No.: PCT/EP2024/025172

§ 371 Date: Nov. 26, 2025

(30) Foreign Application Priority Data
May 30, 2023 (EP) 23305853.6

(51) Int. Cl. A61K 47/69; A61K 38/28; A61K 47/10

(52) U.S. Cl. CPC A61K 47/6951; A61K 38/28; A61K 47/10

(57) ABSTRACT

A composition comprising a cold-water insoluble crosslinked dextrin and a compound selected from 1-decanoyl-rac-glycerol and carboxymethyl cellulose. This invention also relates to a method for making such composition. Also disclosed is the use of a combination of a cold-water insoluble crosslinked dextrin and of a compound selected from 1-decanoyl-rac-glycerol and carboxymethyl cellulose, for increasing the epithelial permeation of an active ingredient, or for the epithelial delivery of an active ingredient.

### TECHNICAL FIELD

The invention relates to the epithelial delivery of poorly permeable drugs, in particular to the oral or intrarectal delivery of poorly permeable drugs such as biopharmaceuticals, for example therapeutic proteins.

### BACKGROUND ART

There is both great interest and a medical need for improving the oral bioavailability of various poorly bioavailable drugs. Maximizing oral bioavailability is therapeutically important because the extent of bioavailability directly influences plasma concentrations, as well as the therapeutic and toxic effects, resulting after oral drug administration. Poorly bioavailable drugs are inefficient because a major portion of a dose never reaches the plasma or exerts its pharmacologic effect. Moreover, a compilation of published results on structurally diverse drugs showed that the intersubject variability in bioavailability was inversely correlated with the extent of bioavailability. Therefore, low oral bioavailability leads to high variability and poor control of plasma concentrations and effects

Incomplete oral bioavailability has various causes. These include poor dissolution or low aqueous solubility, degradation of the drug in gastric or intestinal fluids, poor intestinal membrane permeation, and presystemic intestinal or hepatic metabolism.

Some physicochemical properties that have been associated with poor membrane permeability are low octanol/aqueous partitioning, the presence of strongly charged functional groups, high molecular weight, a substantial number of hydrogen-bonding functional groups, and high polar surface area. The compounds that may benefit most from intestinal absorption enhancing formulations usually have one or more of these characteristics. Typically, the drugs involved in absorption enhancement studies have been proteins, peptides, peptide analogs, or other polar, high molecular weight drugs, such as heparin.

Among the strategies that have been developed for improving intestinal permeation of those drugs, mention can be made of the use of the following permeation enhancers:
surfactants (e.g., polyoxyethylene (POE) ethers, POE esters, POE sorbitan esters, dodecylmaltoside, nonylphenoxypolyoxyethylene surfactants, sucrose laurate)
fatty acids (e.g., sodium decanoate, caprylic acid, capric acid, oleic acid, linoleic acid, linolenic acid);
medium-chain glycerides (e.g., monoglycerides and diglycerides of caprylic and capric acid, monohexanoin), eventually combined with emulsifying or solubilizing agents;
steroidal detergents (e.g., bile salts (sodium taurocholate), chenodeoxycholate, ursodeoxycholate, saponins, glycyrrhizinate, glycyrrhetinic acid, glycosylated bile acid analogs);
acylcarnitines and alkanoylcholines (e.g., medium and long chain fatty acid esters of carnitine and choline, for example Palmitoyl-DL-carnitine chloride, lauroyl choline);
N-acetylated α-amino acids and N-acetylated Non-α-amino acids (e.g., N-cyclohexanoylleucine, N-(phenylsulphonyl)leucine, 4-[4-[(2-hydroxybenzoyl)amino]phenyl]butyric acid, N-[8-(2-hydroxybenzoyl)amino]caprylate (SNAC));
chitosans and other mucoadhesive polymers (e.g., the anionic polyacrylic acid derivatives, polycarbophil and carbomer (i.e., Carbopol 934P), cationic chitosans, trimethyl chitosan);
secretory transport inhibitors (e.g., P-glycoprotein (Pgp) inhibitors, for example SDZ PSC 833, cyclosporin, Polysorbate 80, POE 35 castor oil (Cremophor EL®), poloxamers (Pluronic P85®);
cyclodextrins.

There is however still a need for additional or more efficient intestinal permeation enhancer.

The development of a permeation enhancer useful for intestinal permeation would allow increasing the bioavailability of poorly permeable drugs taken orally, but also of those taken intrarectally. It may also find applications in other epithelial routes that are administration routes requiring the active ingredient to cross the epithelium such as cutaneous route, mucosal route (e.g., vaginal route, sublingual route, buccal route), transdermal route, ophthalmic route, nasal route, transnasal route, or bronchopulmonary route.

### Technical Problem

It was an object of the present invention to provide an improved intestinal permeation enhancer, and more broadly an improved epithelial permeation enhancer, for poorly permeable active ingredients such as of biopharmaceuticals.

It was an object of the present invention to provide a material able to improve the bioavailability of active ingredients such as of biopharmaceuticals when taken orally or intrarectally, and more broadly when taken by epithelial route.

It was an object of the present invention to solve this technical problem by offering a solution that has the other properties required for its intended purpose, for example in terms of purity and safety.

### PRESENTATION OF THE INVENTION

The inventor(s) solved the above-mentioned problem by providing an epithelial (e.g., intestinal) permeation enhancer which is a combination of:
a cold-water insoluble crosslinked dextrin; and,
a compound selected from 1-decanoyl-rac-glycerol and carboxymethyl cellulose (CMC).

As it is apparent form the Example section herein after, these substances seem to act in synergy, thus allowing to obtain a high enhancement of intestinal permeation.

The results obtained on Caco-2 cell model make it a promising permeation enhancer for intestinal routes such as oral and intrarectal routes. It also makes it a promising permeation enhancer for other epithelial routes such as cutaneous route, mucosal route (e.g., vaginal route, sublingual route, buccal route), transdermal route, ophthalmic route, nasal route, transnasal route, or bronchopulmonary route.

### BRIEF DESCRIPTION OF THE INVENTION

The disclosure first relates to a composition comprising:
a cold-water insoluble crosslinked dextrin; and,
a compound selected from 1-decanoyl-rac-glycerol and carboxymethyl cellulose (CMC).

Preferably, said dextrin is selected from pyrodextrins, maltodextrins, cyclodextrins, or from a mixture thereof. Preferably, said cold-water insoluble crosslinked dextrin is obtainable by reacting a dextrin with a crosslinker, said crosslinker being selected from trimetaphosphate salts, dicarboxylic acids, dianhydrides, carbonyldiimidazole, diphenylcarbonate, triphosgene, acylic dichlorides, diisocyanates, diepoxides, or from any mixture thereof. Preferably, said cold-water insoluble crosslinked dextrin is obtainable by reacting a dextrin with a crosslinker, wherein the molar ratio of said crosslinker to the anhydroglucose units of said dextrin is equal to or higher than 0.1:1. Preferably, said cold-water insoluble crosslinked dextrin is in the form of particles.

The disclosure also relates to a composition comprising:
a cold-water insoluble crosslinked dextrin; and,
a compound selected from 1-decanoyl-rac-glycerol and carboxymethyl cellulose (CMC); and,
an active ingredient.

Preferably, said active ingredient is selected from BCS class III drugs, BCS class IV drugs, biopharmaceuticals, or from any mixture thereof. Preferably, said active ingredient is selected from proteins. Preferably, said active ingredient is loaded in said cold-water insoluble crosslinked dextrin.

The disclosure also relates to such composition for use as a medicament and to the use of such composition in a food composition such as a food supplement, or in a nutraceutical, or cosmetic composition.

Preferably, the compositions according to the disclosure are intended to be administered by epithelial route, preferably by a route selected from intestinal route (including oral or intrarectal routes), cutaneous route, mucosal route (e.g., vaginal route, sublingual route, buccal route), transdermal route, ophthalmic route, nasal route, transnasal route, or bronchopulmonary route. Preferably, the compositions according to the disclosure are intended to be administered by intestinal route, preferably by oral or intrarectal route, more preferably by oral route. In other words, they are preferably oral or intrarectal compositions, more preferably oral compositions.

The term “epithelial routes” classically refers to routes of administration requiring the crossing of an epithelium. It may be a systemic route, but also a local route of administration (like this is the case for example of cutaneous administration of active ingredients requiring crossing the epidermis to reach the dermis). It is still preferably a systemic route of administration.

The disclosure also relates to a method for the preparation of such compositions, comprising putting into contact said cold-water insoluble crosslinked dextrin with said compound selected from 1-decanoyl-rac-glycerol and carboxymethyl cellulose (CMC).

The disclosure also relates to the use of a combination:
of a cold-water insoluble crosslinked dextrin,
and of a compound selected from 1-decanoyl-rac-glycerol and carboxymethyl cellulose (CMC),
   for increasing the epithelial permeation of an active ingredient, preferably for increasing the intestinal permeation of an active ingredient.

The disclosure also relates to the use of a combination:
of a cold-water insoluble crosslinked dextrin,
and of a compound selected from 1-decanoyl-rac-glycerol and carboxymethyl cellulose (CMC),
   for the epithelial delivery of an active ingredient, preferably for the oral or intrarectal delivery of an active ingredient, more preferably for the oral delivery of an active ingredient.

Preferably, said active ingredient is selected from BCS class Ill drugs, BCS class IV drugs, biopharmaceuticals, or from any mixture thereof. Preferably, said active ingredient is selected from proteins.

### BRIEF DESCRIPTION OF DRAWINGS

Other features, details and advantages will be shown in the following detailed description and on the figures, on which:

FIG. 1 a scheme of the 24-well Caco-2 model (Apical to basolateral passage only).

FIG. 2 is a bar chart showing the permeation of insulin when using a combination of a cold-water insoluble crosslinked dextrin (nanosponges) and of 1-decanoly-rac-glycerol.

FIG. 3 is a bar chart showing the permeation of insulin when using a combination of a cold-water insoluble crosslinked dextrin (nanosponges) and of carboxymethyl cellulose (CMC).

FIG. 4 is a bar chart showing the permeation of insulin when using a combination of a cold-water insoluble crosslinked dextrin (nanosponges) and of salcaprozate sodium.

FIG. 5 is a bar chart showing the permeation of insulin when using a combination of a cold-water insoluble crosslinked dextrin (nanosponges) and of lauryl-L-carnitine.

FIG. 6 is a bar chart showing the permeation of insulin when using a combination of a cold-water insoluble crosslinked dextrin (nanosponges) and of nonaethylen glycol monododecyl ether.

FIG. 7 is a bar chart showing the permeation of insulin when using a combination of a cold-water insoluble crosslinked dextrin (nanosponges) and of polyethylene glycol.

FIG. 8 is a bar chart showing the permeation of insulin when using a combination of a cold-water insoluble crosslinked dextrin (nanosponges) and of Silica, monodisperse 20 μm.

FIG. 9 is a bar chart showing the permeation of insulin when using a combination of a cold-water insoluble crosslinked dextrin (nanosponges) and of Silica, monodisperse 150 nm.

FIG. 10 is a bar chart showing the permeation of insulin when using a combination of a cold-water insoluble crosslinked dextrin (nanosponges) and of kaolinite.

FIG. 11 is a bar chart showing the permeation of insulin when using a combination of a cold-water insoluble crosslinked dextrin (nanosponges) and of montmorillonite.

### DESCRIPTION OF EMBODIMENTS

Figures and the following detailed description contain, essentially, some exact elements. They can be used to enhance the understanding of the invention and, also, to define the invention if necessary.

Composition Comprising a Cold-Water Insoluble Crosslinked Dextrin and a Compound Selected from 1-Decanoyl-Rac-Glycerol and CMC

The invention first relates to a composition comprising:
a cold-water insoluble crosslinked dextrin; and,
a compound selected from 1-decanoyl-rac-glycerol and CMC.

The term “dextrin” classically refers to products obtained from starch hydrolysis, including maltodextrins, glucose syrups having a dextrose equivalent (DE) from 20 to 30, pyrodextrins and cyclodextrins. Preferably, the dextrin according to the disclosure is selected from maltodextrins, pyrodextrins, cyclodextrins, or from any mixture thereof. It is still preferably selected from maltodextrins, pyrodextrins, or from any mixture thereof. It is still preferably selected from maltodextrins or from any mixture thereof i.e., it is preferably a cold-water insoluble crosslinked maltodextrin.

The term “maltodextrin” classically refers to a dextrin obtained by acid and/or enzymatic hydrolysis of starch. Maltodextrins are typically characterized by a DE lower than 20. Preferably, the maltodextrins according to the disclosure have a DE equal to or higher than 1, preferably equal to or higher than 5, preferably equal to or higher than 10, preferably equal to or higher than 15. It is preferably equal to or lower than 19. It is for example equal to about 17.

The term “pyrodextrin” classically refers to a dextrin obtained by dry heating of starch under acidic conditions, which generally leads to hydrolysis of the starch followed by reconnection via α-1,6 bonds. In general, these pyrodextrins are classified in “white dextrins”, “yellow dextrins”, or “British gum”, depending on the temperature, acidity and humidity conditions used.

The term “cyclodextrin” refers to cyclic dextrins, native or substituted, having 6 to 12 glucose units bounded to one another generally via carbons C1 and C4. Cyclodextrins include α-, β- and γ-cyclodextrins having 6, 7 and 8 glucose units respectively. Preferably, the cyclodextrin according to the disclosure is selected from α-cyclodextrins, β-cyclodextrins, γ-cyclodextrins, or from any mixture thereof, still preferably from β-cyclodextrins. The cyclodextrins according to the disclosure may be substituted, in particular etherified, preferably with alkyl and/or hydroxyalkyl and/or sulfoalkyl groups. Preferably, the alkyl, hydroxyalkyl and sulfoalkyl groups according to the disclosure have 1 to 5 carbon atoms, preferably 1 to 4, preferably 1 or 3 or 4. Preferably, the substituted cyclodextrin according to the disclosure is selected from methyl-cyclodextrins, hydroxypropyl-cyclodextrins, sulfobutylether-cyclodextrins, or from any mixture thereof. Contrary to chemical substance having well defined structure, substituted cyclodextrins generally are a mixture of substituted cyclodextrins having different substitution patterns, and which are thus structurally different. More preferably, the cyclodextrin according to the disclosure is unsubstituted cyclodextrin (i.e., native cyclodextrin), preferably unsubstituted β-cyclodextrin (i.e., native β-cyclodextrin).

Dextrins are classically obtained from starch. The term “starch” classically refers to starch isolated from any suitable botanical source, by any technique well known to those skilled in the art. Isolated starch typically contains no more than 3% of impurities; said percentage being expressed in dry weight of impurities with respect to the total dry weight of isolated starch. These impurities typically comprise proteins, colloidal matters and fibrous residues. Suitable botanical source includes for instance legumes, cereals, and tubers.

The dextrin according to the disclosure can thus be selected from legume dextrins (e.g., pea dextrins, fava bean dextrins), cereal dextrins (e.g., maize dextrins, rice dextrins, wheat dextrins, oat dextrins), and tuber dextrins (e.g., potato dextrins, tapioca dextrins). Preferably, the dextrin according to the disclosure are selected from legume dextrins, cereal dextrins, or from any mixture thereof.

Preferably, the legume dextrin is selected from pea dextrins, fava bean dextrins, or from any mixture thereof, still preferably from pea dextrins. The term “pea” classically include: all wild and mutant varieties of “smooth pea” and of “wrinkled pea”, regardless of the uses for which the varieties are generally intended for (human food, animal feed and/or other uses). The term “pea” includes pea of Pisum genius, more specifically of sativum and aestivum species. The mutant varieties may be those referred to as “r mutants”, “rb mutants”, “rug 3 mutants”, “rug 4 mutants”, “rug 5 mutants” and “lam mutants” as described in the article by C-L HEYDLEY et al. entitled “Developing novel pea starches”, Proceedings of the Symposium of the Industrial Biochemistry and Biotechnology Group of the Biochemical Society, 1996, pages 77-87. Preferably, the dextrins according to the disclosure are selected from smooth pea dextrins, still preferably from wild smooth pea dextrins.

Preferably, the cereal dextrin is a maize dextrin.

Preferably, when the dextrin according to the disclosure is a maltodextrin, it is a legume maltodextrin, preferably a pea maltodextrin, preferably a smooth pea maltodextrin, preferably a wild smooth pea maltodextrin.

Preferably, when the dextrin according to the disclosure is a pyrodextrin, it is a cereal pyrodextrin, preferably a maize pyrodextrin.

In a preferred embodiment, the dextrin according to the disclosure is obtained from a starch having an amylose content equal to or higher than 20%, preferably equal to or higher than 25%, preferably equal to or higher than 30%, preferably equal to or higher than 35%; this content being expressed in dry weight with respect to the total dry weight of starch. This amylose content is preferably equal to or lower than 90%, preferably equal to or lower than 60%, preferably equal to or lower than 50%, preferably equal to or lower than 45%, preferably equal to or lower than 40%, preferably equal to or lower than 38%. Preferably, this starch is a legume starch, preferably a pea or a fava bean starch, still preferably a smooth pea starch, still preferably a wild smooth pea starch. This amylose content can be determined by the person skilled in the art by way of potentiometric analysis of iodine absorbed by amylose to form a complex.

Preferably, the dextrin according to the disclosure, in particular the maltodextrin according to the disclosure, has a weight average molecular weight (Mw), determined by liquid chromatography with detection by differential refractometer, equal to or higher than 1 000 Da, preferably equal to or higher than 5 000 Da, preferably equal to or higher than 8 000 Da, preferably equal to or higher than 10 000 Da. This Mw is preferably equal to or lower than 200 000 Da, preferably equal to or lower than 100 000 Da, preferably equal to or lower than 50 000 Da, preferably equal to or lower than 40 000 Da, preferably equal to or lower than 30 000 Da, preferably equal to or lower than 20 000 Da, preferably equal to or lower than 15 000 Da. It is for example equal to about 12 000 Da.

This Mw is determined by liquid chromatography with detection by differential refractometer, preferably using pullulan standards. It may be determined by the person skilled in the art according to the following protocol:

The following set of columns (e.g., Shodex OH pak SB-800 QH) is used, in the following order:
a column with a particle size of 8 μm, a pore size of 100 Å, an inner diameter of 8.0 mm, a length of 300 mm (e.g., OH pak SB-802 HQ—Waters réf. JWE 034256);
a column with a particle size of 6 μm, a pore size of 800 Å, an inner diameter of 8.0 mm, a length of 300 mm (e.g., OH pak SB-803 HQ—Waters réf. JWE 034257);
a column with a particle size of 13 μm, a pore size of 7 000 Å, an inner diameter of 8.0 mm, a length of 300 mm (e.g., OH pak SB-805 HQ—Waters réf. JWE 034259). Pulluan standards (e.g., Kit Waters-Ref. JWE034207) with the following Mw are used: P800, P400, P200, P100, P50, P20, P10, P5.
   Conditions: eluting solvent: aqueous solution of sodium nitrate 0.1M containing 0.02% sodium azide filtrated on a 0.02 μm filter; flow of the mobile phase: 0.5 mL/min; column temperature: 35 °C; injected volume: 100 μL; detector: RI attenuation: 16, inner temperature: 35 °C; analysis time: 80 minutes.
   Calibration with Glycerin Mw=92; Glucose Mw=180; Maltose Mw=342; Maltotriose Mw=504; Maltotetraose Mw=667; Maltopentaose Mw=829; Maltohexaose Mw=991; Maltoheptaose Mw=1153.

Examples of suitable dextrins are commercially available. Mention can be made for example of the following products marketed by ROQUETTE: KLEPTOSE® β-cyclodextrin, KLEPTOSE® Linecaps (pea maltodextrin), GLUICIDEX®2 (waxy maize pyrodextrin), STABILYS® A025 (maize pyrodextrins), STABILYS® A053 (maize pyrodextrins), and TACKIDEX® range.

The cold-water insoluble dextrin according to the disclosure is crosslinked, meaning that it is obtainable by (or obtained by) reacting a dextrin with a crosslinker. The crosslinker typically is polyfunctional i.e., the crosslinker comprises at least 2 reactive groups. Preferably, the crosslinker is bifunctional. Typically, in the instant disclosure, the reactive groups have an atom subjected to nucleophilic attack i.e., having a partial positive charge.

Preferably, the crosslinker according to the disclosure is selected from trimetaphosphate salts, dicarboxylic acids, dianhydrides, carbonyldiimidazole, diphenylcarbonate, triphosgene, acylic dichlorides, diisocyanates, diepoxides, or from any mixture thereof. Preferably, the dicarboxylic acids are selected from polyacrylic acid, butane tetracarboxylic acid, succinic acid, tartaric acid, citric acid, or from a mixture thereof. It is still preferably selected from citric acid and/or tartaric acid. Preferably, the dianhydrides are selected from diethylenetriaminepentaacetic dianhydride, ethylenediaminetetraacetic dianhydride, benzophenone-3,3′,4,4′-tetracarboxylic dianhydride, pyromellitic dianhydride, or from any mixture thereof. It is still preferably pyromellitic dianhydride. Preferably, the acylic chlorides are selected from terephthaloyl chloride, sebacoil chloride, succinyl chloride, or from a mixture thereof. It is still preferably terephthaloyl chloride. Preferably, the diisocyanates are selected from toluendiisocyanate, isophorone diisocyanate, 1,4-phenylene diisocyanate, poly(hexamethylene diisocyanate), hexamethylene diisocyanate, or from a mixture thereof. It is still preferably hexamethylene diisocyanate.

Preferably, the crosslinker according to the disclosure is selected from trimetaphosphate salts, pyromellitic dianhydride, 1,1′-carbonyldiimidazole, hexamethylene diisocyanate, citric acid, tartaric acid, or from a mixture thereof.

In a preferred embodiment, in particular when the dextrin is selected from maltodextrins and/or pyrodextrins, the crosslinker is selected from trimetaphosphate salts. It is still preferably sodium trimetaphosphate.

In a preferred embodiment, in particular when the dextrin is selected from cyclodextrins, the crosslinker is pyromellitic dianhydride.

Preferably, the cold-water insoluble dextrin according to the disclosure is obtainable by (or obtained by) reacting a dextrin with a crosslinker without having undergone a prior step of ethyl-amination (grafting with ethyl-amine groups).

Preferably, the molar ratio of crosslinker to the anhydroglucose units of the dextrin is equal to or higher than 0.1:1, preferably equal to or higher than 0.2:1, preferably equal to or higher than 0.20:1. It is preferably equal to or lower than 10:1, preferably equal to or lower than 5:1, preferably equal to or lower than 4:1, preferably equal to or lower than 3:1, preferably equal to or lower than 2:1, preferably equal to or lower than 1:1, preferably equal to or lower than 1.0:1, preferably equal to or lower than 0.9:1, preferably equal to or lower than 0.8:1, preferably equal to or lower than 0.7:1, preferably equal to or lower than 0.6:1, preferably equal to or lower than 0.5:1, preferably equal to or lower than 0.4:1, preferably equal to or lower than 0.40:1. It is for example equal to about 0.3:1.

In the instant disclosure, it is referred to amounts or ratios of ingredients constituting the cold-water insoluble crosslinked dextrin. It should be understood that these amounts refer to the amounts of starting materials used for making said cold-water insoluble crosslinked dextrin (dextrin, crosslinker, and eventual other ingredients). These amounts may differ a little from the amounts really present in the final matrix (i.e., in the cold-water insoluble crosslinked dextrin prepared therefrom), which, to the best of the inventor(s)′ knowledge, cannot be measured as of today.

The dextrin or crosslinked dextrin according to the disclosure may undergo a step of physical modification, commonly referred to as a “cooking” or “gelatinization” step. This step of cooking is well known to those skilled in the art and is typically performed when the dextrin is at least partially in the granular state. This cooking step allows the polymer molecules constituting the dextrin to be fully and homogeneously dispersed in the reaction solvant (e.g., water). This cooking step is particularly useful when the dextrin is a pyrodextrin. Preferably, the cooking is performed before the crosslinking step.

The crosslinked dextrin according to the disclosure is cold-water insoluble. The term “cold-water” classically refers to water placed at room temperature (in general from 18 to 25 °C). Preferably, the crosslinked dextrin according to the disclosure is insoluble in cold water at a pH of 7. Preferably, the crosslinked dextrin according to the disclosure is insoluble in cold water at a pH of 5. Preferably, the crosslinked dextrin according to the disclosure is insoluble in cold water at a pH of 9.

The crosslinked dextrin according to the disclosure is cold-water insoluble. That is to say that the crosslinked dextrin typically has a solubility in cold water equal to or lower than 20%, this percentage being expressed in dry weight of solubles with respect to the total dry weight of crosslinked dextrin. This solubility is preferably equal to or lower than 15%, preferably, equal to or lower than 10%, preferably equal to or lower than 9%, preferably equal to or lower than 8%, preferably equal to or lower than 7%, preferably equal to or lower than 6%, preferably equal to or lower than 5%, preferably equal to or lower than 4%, preferably equal to or lower than 3%, preferably equal to or lower than 2%, preferably equal to or lower than 1%. This solubility may be determined by the person skilled in the art according to the following protocol: 2.5 g of the product to be tested (e.g., crosslinked dextrin particles for example obtained after grinding and sieving with a 315 μm sieve to eliminate coarse particles) are placed into 150 g of demineralized water, and place under agitation for 16 hours and then centrifugated 15 minutes at 4700 rpm (for example using centrifuge VWR Mega Star 1.6). The supernatant is then placed into a pre-tared crystallizer, and placed under vacuum oven at 55 °C, until there is no more weight loss (all water evaporated). The remaining (solubles) is weighted. This fraction of solubles may comprise a soluble fraction of the crosslinked dextrin, but also some residual impurities.

Preferably, the cold-water insoluble crosslinked dextrin according to the disclosure is in the form of particles. These particles may have an average diameter selected from 1 nm to 1000 μm, for example from 10 nm to 500 μm, said diameter being measured on a suspension in water of said cold-water insoluble crosslinked dextrin. These particles may have an average diameter equal to or lower than 1000 nm (also referred to as “nanosponges”).

It may be used in the form of a nanosuspension of said particles of cold-water insoluble crosslinked dextrin (nanosponges suspension). The suspension may be prepared according to the following protocol:
1. Preparation of a suspension starting from coarse powder in distilled water at the concentration of 10 mg/mL under stirring at room temperature.
2. Dispersion of the suspension using a high shear homogenizer (e.g., Ultraturrax®, IKA, Konigswinter, Germany) for 10 minutes at 24000 rpm.
3. High pressure homogenization for 90 minutes at a back-pressure of 500 bar, (e.g., using an EmulsiFlex C5 instrument (Avastin, USA)) for further size reduction.
4. Purification of homogenized nanosuspension by dialysis, for example using a cellulose membrane with a cutoff of 12000 Da (Spectrapor) to remove synthesis residues potentially present.
5. If required, storage of the nanosuspensions at 4 °C.

Preferably, the particles of cold-water insoluble crosslinked dextrin according to the disclosure have an average diameter of 1 to 1000 nm. It is preferably equal to or higher than 10 nm, preferably equal to or higher than 50 nm, preferably equal to or higher than 100 nm. This average diameter is a hydrodynamic diameter. It can be determined by the person skilled in the art by Laser Light Scattering, for example using the 90Plus Instrument (Brookhaven, NY, USA), preferably on a nanosponges suspension, for example obtained according to the protocol given above, said suspension comprising 10 mg/ml of nanosponges being diluted with filtered (0.22 μm) distilled water, using a dilution factor of 1/30 by volume.

Preferably, the polydispersity index relative to said average diameter is lower than 1.00. This polydispersity index is in general higher than 0.01, even equal to or higher than 0.05.

Preferably, the cold-water insoluble dextrin according to the disclosure has a zeta potential lower than 0 mV, preferably equal to or lower than-10 mV, preferably equal to or lower than-20 mV. It is in general equal to or higher than-50 mV, even equal to or higher than-40 mv. This zeta potential can be determined by the person skilled in the art using electrophoretic mobility by dynamic light scattering, at a scattering angle of 90° at a temperature of 25 °C, on a crosslinked dextrin suspension, for example using the 90Plus Instrument (Brookhaven, NY, USA). The sample is placed in the electrophoretic cell, where an electric field of 15V/cm is applied. Preferably, this zeta potential is determined on a nanosponges suspension, for example obtained according to the protocol given before, the suspension comprising 10 mg/ml of nanosponges being diluted with filtered (0.22 μm) distilled water, using a dilution factor of 1/30 by volume.

Preferably, the cold-water insoluble crosslinked dextrin according to the disclosure has a swelling index (SI %) equal to or higher than 200%, preferably equal to or higher than 500%, preferably equal to or higher than 600%, preferably equal to or higher than 700%, preferably equal to or higher than 800%, preferably equal to or higher than 900%, preferably equal to or higher than 1000%, preferably equal to or higher than 1100%, preferably equal to or higher than 1200%, preferably equal to or higher than 1300%, preferably equal to or higher than 1400%, preferably equal to or higher than 1500%, preferably equal to or higher than 1600%. It is preferably equal to or lower than 5000%, preferably equal to or lower than 4000%, preferably equal to or lower than 3000%, preferably equal to or lower than 2000%.

The swelling index of a matrix (e.g., cold-water insoluble dextrin according to the disclosure) is defined by the following formula:

### [Math]

$$SI \% = \frac{{W}_{s} - {W}_{d}}{{W}_{d}} \times 10$$

wherein Wd is the weight of matrix and Ws is the weight of swollen matrix.

For determining this SI %, 1 g (dry weight) of matrix (after grinding and sieving with a 315 μm sieve to eliminate coarse particles) is dispersed in 100 ml demineralized water in a graduated cylinder, and left 24 hours for swelling. After 24 hours of contact, the mixture of matrix dispersed in water is centrifuged to separate the supernatant (water) and the bottom layer (swollen matrix or gel). The swollen matrix is then weighed.

The dextrin according to the disclosure might undergo other chemical and/or physical modifications than the preferred ones described before (i.e., crosslinking, and eventually cooking), as long as it does not interfere with the desired properties, notably in term of safety and efficiency. However, and because it is not necessary to solve the technical problem disclosed herein, the dextrin according to the disclosure is preferably no further modified.

The cold-water insoluble crosslinked dextrin according to the disclosure might include other ingredients in its structure than the dextrin and the crosslinking pattern derived from the crosslinker, as long as it does not interfere with the desired properties of said cold-water insoluble crosslinked dextrin, in particular in terms of efficiency and safety. It is understood that the term “other ingredients” does not refer to the small amounts of impurities eventually brought by the dextrin and the crosslinker. Examples of such other ingredients are other polymers, for example proteins, which are typically also crosslinked if used.

However, and because it is not necessary to solve the technical problem disclosed herein, the cold-water insoluble crosslinked dextrin according to the disclosure preferably has an amount of said other ingredients equal to or lower than 30%, preferably equal to or lower than 20%, preferably equal to or lower than 10%, preferably equal to or lower than 5%, preferably equal to or lower than 1%, preferably equal to 0%; said percentage being expressed in dry weight, with respect to the total dry weight of said cold-water insoluble dextrin. Still preferably, the cold-water insoluble crosslinked dextrin according to the disclosure includes no other ingredients.

Therefore, in a preferred embodiment, the cold-water insoluble crosslinked dextrin according to the disclosure consists in a crosslinked dextrin. Preferably, the dextrin is selected from maltodextrins, pyrodextrins, cyclodextrins, or from any mixture thereof. Still preferably, the cold-water insoluble dextrin consists in a crosslinked maltodextrin, or in a crosslinked pyrodextrin, or in a crosslinked cyclodextrin.

Examples of suitable cold-water insoluble crosslinked dextrin as well as processes for making thereof are described in patents applications WO 2016/004974 A1 (ROQUETTE) and WO 2021/254662 A1 (ROQUETTE).

The composition according to the disclosure also comprises a compound selected from 1-decanoyl-rac-glycerol and CMC.

The CMC may be in the form of a salt or in a protonated form. It is preferably a salt of CMC. It is still preferably sodium CMC.

Preferably, the dry weight ratio of 1-decanoyl-rac-glycerol to cold-water insoluble crosslinked dextrin is from 0.05:1 to 5:1. It is preferably equal to or higher than 0.10:1. It is preferably equal to or lower than 4:1, preferably equal to or lower than 3:1, preferably equal to or lower than 2:1, preferably equal to or lower than 1:1, preferably equal to or lower than 1.0:1, preferably equal to or lower than 0.9:1, preferably equal to or lower than 0.8:1, preferably equal to or lower than 0.7:1, preferably equal to or lower than 0.6:1, preferably equal to or lower than 0.5:1, preferably equal to or lower than 0.4:1, preferably equal to or lower than 0.3:1, preferably equal to or lower than 0.2:1, preferably equal to or lower than 0.20:1. It is for example equal to about 0.15:1.

Preferably, the dry weight ratio of CMC to cold-water insoluble crosslinked dextrin is from 0.05:1 to 5:1. It is preferably equal to or higher than 0.10:1, preferably equal to or higher than 0.2:1, preferably equal to or higher than 0.3:1, preferably equal to or higher than 0.4:1, preferably equal to or higher than 0.5:1, preferably equal to or higher than 0.6:1, preferably equal to or higher than 0.7:1, preferably equal to or higher than 0.8:1, preferably equal to or higher than 0.9:1, preferably equal to or higher than 1.0:1, preferably equal to or higher than 1.1:1, preferably equal to or higher than 1.2:1, preferably equal to or higher than 1.20:1. It is preferably equal to or lower than 4:1, preferably equal to or lower than 3:1, preferably equal to or lower than 2:1, preferably equal to or lower than 1.5:1, preferably equal to or lower than 1.4:1, preferably equal to or lower than 1.3:1, preferably equal to or lower than 1.30:1. It is for example equal to about 1.25:1.

The invention also relates to the use of a composition according to the disclosure in a medicament or in a food composition such as a food supplement, or in a nutraceutical or cosmetic composition.

The composition according to the disclosure may be a product intended to be administered to an individual or a composition useful for the preparation of compositions intended to be administered to an individual. It may be a powdery composition, a tablet or a suspension. Said powdery composition may be administered as is, eventually with the addition of water. It might also be encapsulated, for example into hard capsules. Said suspension might be administered as is or encapsulated for example into soft capsules.

Composition Comprising a Cold-Water Insoluble Crosslinked Dextrin, a Compound Selected from 1-Decanoyl-Rac-Glycerol and CMC and an Active Ingredient

The combination of cold-water insoluble crosslinked dextrin and of 1-decanoyl-rac-glycerol or CMC according to the disclosure allows to increase the intestinal permeation (and more broadly epithelial permeation) of active ingredients.

The instant disclosure thus also relates to a composition comprising:
a cold-water insoluble crosslinked dextrin; and,
a compound selected from 1-decanoyl-rac-glycerol and CMC; and,
an active ingredient.

Preferably, said composition is as described before. Preferably, said cold-water insoluble crosslinked dextrin is as described before.

The term “active ingredient” classically refers to any substance of pharmaceutical, veterinary, food, nutraceutical, or cosmetic interest. Preferably, the active ingredient according to the disclosure, is a pharmaceutical, nutraceutical, cosmetic or veterinary active ingredient, still preferably a pharmaceutical active ingredient. The active ingredient according to the disclosure, in particular the pharmaceutical active ingredient, may be selected from so-called small molecules or from so-called large molecules (also referred to as “biopharmaceuticals”), for example from proteins, nucleic acids, viruses, cells. Biopharmaceuticals are typically not bioavailable when taken orally, for example because this administration route causes their degradation and/or because they are not able to cross biological membranes (including the epithelium). In other words, these are typically the active ingredients that need to be administered parenterally and for which a systemic effect is sought. Non limitative examples of biopharmaceuticals are vaccines, blood components (for example coagulation factors, blood fractionation products), antibodies (e.g., monoclonal antibodies, humanized antibodies, chimeric antibodies, monoclonal antibodies, antibodies fragments e.g., variable fragments of antibodies), allergens, hormones such as insulin, gene therapy agents, tissues, cell therapy agents, recombinant therapeutic proteins. Preferably, the biopharmaceutical according to the disclosure is selected from proteins. The term “protein” should be understood broadly. It notably encompasses proteins regardless their manufacturing process or their number of subunits. It also encompasses protein fragments, peptides, oligopeptides. It may be selected from native proteins, recombinant proteins, fusion proteins or from any mixture thereof. It is understood that when a protein is derived from a natural product e.g., form a vegetal, it is preferably an isolated protein.

Preferably, the protein according to the disclosure has at least 5 amino acids, preferably at least 10, preferably at least 20, preferably at least 30, preferably at least 40, preferably at least 50. Preferably, the protein according to the disclosure has a maximum of 5 000 amino acids, preferably a maximum of 1 000, preferably a maximum of 500, preferably a maximum of 400, preferably a maximum of 300, preferably a maximum of 200, preferably a maximum of 100, preferably a maximum of 90, preferably a maximum of 80, preferably a maximum of 70, preferably a maximum of 60.

Preferably, the protein according to the disclosure (e.g., the pharmaceutical protein), is selected from enzymes, cytokines, hormones, growing factors, plasmatic factors, vaccines, antibodies. It is preferably insulin. The term “insulin” encompasses insulin or any pharmaceutically active derivative thereof, preferably insulin.

Preferably, the active ingredient according to the disclosure is an active ingredient whose epithelial (preferably intestinal) permeation needs to be increased. It is preferably selected from BCS class III and/or IV drugs (Biopharmaceutics Classification System according to the US Food and Drug Administration as in force on Nov. 1, 2022).

Non-limiting examples of active ingredients whose epithelial (in particular intestinal) permeation needs to be increased are poorly absorbed antibiotics such as erythromycin, colistin, cefamandole, cefotaxime, moxalactam, mezlocillin, penicillin G, ampicillin, cefoxitin, carumonam gentamicin, vancomycin; octreotide; calcitonin; cromolyn; insulin; glucagon; recombinant human growth hormone; doxorubicin, paclitaxel, etoposide, azidodeoxythymidine, argvasopressin. Preferably, the active ingredient according to the disclosure is a biopharmaceutical, still preferably a protein, still preferably insulin.

Preferably, the active ingredient according to the disclosure is an active ingredient whose absorption can be increased by transportation through tight junctions.

Preferably, the active ingredient according to the disclosure is intended to be administered by a route selected from intestinal route (including oral or intrarectal routes), cutaneous route, mucosal route (e.g., vaginal route, sublingual route, buccal route), transdermal route, ophthalmic route, nasal route, transnasal route, or bronchopulmonary route. Preferably, the active ingredient according to the disclosure is intended to be administered by intestinal route, preferably by intrarectal or oral route. More preferably, it is intended to be administered orally.

Preferably, the active ingredient is loaded in the cold-water insoluble crosslinked dextrin according to the disclosure. Insofar as the nature of the association between the crosslinked dextrin and the active ingredient is unknown, the term “loaded” is intended to mean “associated with”, whether inside the matrix formed by the cross-linked dextrin, or on its surface.

The loading may be performed by adding the active ingredient to a pre-formed nanosponges suspension, for example prepared as described before. The mixture is then stirred, for example at room temperature for 30 minutes, for the active ingredient to be incorporated. The media is then centrifuged, and the sediment is collected. It can be lyophilized for future uses.

Preferably, the loading capacity of the cold-water insoluble crosslinked dextrin according to the disclosure is equal to or higher than 1%, this percentage being expressed in dry weight of active ingredient with respect to the total dry weight of the loaded cold-water insoluble crosslinked dextrin. This loading capacity is preferably equal to or higher than 5%, preferably equal to or higher than 10%, preferably equal to or higher than 15%. It is in general equal to or lower than 50%, even equal to or lower than 40%, even equal to or lower than 30%, even equal to or lower than 20%. This loading capacity may be determined by the person skilled in the art according to the following protocol: the loading capacity is determined from freeze-dried loaded samples, preferably prepared as described above. Briefly, a weighted amount of 2-3 mg of freeze-dried delivery system loaded with the active ingredient is dispersed in 5 mL of distilled water. Sonication (15 minutes, 100 W) and centrifugation treatments are performed so as to allow the release of the active ingredient from the crosslinked dextrin. Then the supernatant is analyzed for the quantitative determination of the active ingredient. The loading capacity of the delivery systems is calculated as follows: [dry weight of active ingredient/dry weight of freeze-dried loaded sample]×100.

The composition comprising an active ingredient according to the disclosure may be a dosage form i.e., a product intended to be administered to an individual or a composition useful for the preparation of a dosage form. It may be a powdery composition, a tablet or a suspension. Said powdery composition may be administered as is, eventually with the addition of water. It might also be encapsulated, for example into hard capsules. Said suspension might be administered as is or encapsulated for example into soft capsules.

In a preferred embodiment, the composition according to the disclosure is a suspension.

### Uses of the Compositions According to the Disclosure

The invention also relates to a composition according to the disclosure comprising an active ingredient, for use as a medicament and to the use of a composition according to the disclosure in a food composition such as a food supplement, or in a nutraceutical, or cosmetic composition. In the context of the invention, the use as a medicament is intended to be a human or veterinary use preferably human.

The invention also relates to a method for treating or preventing a disease in an organism in need thereof, comprising administering a composition according to the disclosure comprising an active ingredient to said organism.

The invention also relates to a method for feeding an organism in need thereof, comprising administering a composition according to the disclosure comprising an active ingredient to said organism.

Preferably, said medicaments and compositions are as described before. In particular, said active ingredient is preferably loaded in cold-water insoluble crosslinked dextrin.

Preferably, said medicaments and compositions are intended to be administered by a route selected from intestinal route (including oral or intrarectal routes), cutaneous route, mucosal route (e.g., vaginal route, sublingual route, buccal route), transdermal route, ophthalmic route, nasal route, transnasal route, or bronchopulmonary route. Preferably, said medicaments and compositions are intended to be administered by intestinal route, preferably by intrarectal or oral route. More preferably, it is intended to be administered orally.

Therefore, preferably, said medicaments and compositions are oral or intrarectal compositions, in particular for the oral or intrarectal administration of said active ingredient respectively. More preferably, said medicaments and compositions are oral compositions, in particular for the oral administration of said active ingredient.

Preferably, the disease to be treated in the present disclosure is diabetes, preferably insulin-dependent diabetes, still preferably type-1 diabetes and/or gestational diabetes.

Preferably, the compositions according to the disclosure are for an organism suffering from diabetes, preferably insulin-dependent diabetes, still preferably type-1 diabetes and/or gestational diabetes. Preferably, said organism is a human or an animal, preferably a mammal, still preferably a human.

Preferably, the compositions according to the disclosure, are compositions intended to be administered by epithelial route. They are preferably oral or intrarectal compositions, more preferably oral compositions i.e., compositions intended to be taken orally. They can be compositions intended to be administered as is e.g., dosage forms comprising an active ingredient, or compositions useful for the preparation of compositions intended to be administered as is e.g., dosage forms. It is preferably intended to be administered to a human or an animal, preferably to mammals, still preferably to humans. Preferably, the composition comprising an active ingredient according to the disclosure is a medicament, or a food composition, or a nutraceutical composition, or a cosmetic composition.

### Other Ingredients

The compositions according to the disclosure may include other ingredients, as long as it does not interfere with the desired properties of said compositions, in particular in terms of efficiency and safety. If the composition is a dosage form, these further ingredients will typically depend on the final galenic form. Non-limiting examples of such other ingredients are: binders and fillers (e.g., lactose, microcrystalline cellulose, mannitol), (super)disintegrant (e.g., sodium starch glycolate, crospovidone, croscarmellose), minerals, granulating agent (e.g., polyvinylpyrrolidone, cellulose derivatives, acacia gum, dextrose, gelatin, maltodextrins, starches, starch derivatives, tragacanth gum), flavors, colors, glidants (e.g., silica dioxide), anti-sticking agents (talc), lubricants (e.g., magnesium stearate), solvent (preferably water), buffers, other permeation enhancers. However, and because it is not necessary to obtain the desired effect in the present disclosure, the compositions according to the disclosure preferably include no further permeation enhancers.

The invention thus also relates to a composition consisting of:
a cold-water insoluble crosslinked dextrin, preferably a cold-water insoluble crosslinked maltodextrin; and,
a compound selected from 1-decanoyl-rac-glycerol and CMC; and,
optionally an active ingredient; and,
optionally other ingredients.

Preferably, said cold-water insoluble crosslinked dextrin is as described before. Preferably, said active ingredient is as described before. It is typically loaded in the cold-water insoluble crosslinked dextrin. Preferably, said further ingredients are as described before. Preferably, the amounts of ingredients are as described before.

### Method for Making the Compositions According to the Disclosure

The invention also relates to a method for the preparation of a composition according to the disclosure, comprising putting into contact a cold-water insoluble crosslinked dextrin with a compound selected from 1-decanoyl-rac-glycerol and CMC. The invention also relates to a composition obtained or obtainable by said method.

Preferably, the composition is as described before. Preferably, said cold-water insoluble crosslinked dextrin is as described before.

When the composition is a composition comprising an active ingredient, the method may advantageously include a step of loading (or “associating”) said active ingredient in said cold-water insoluble crosslinked dextrin. For performing the loading, the cold-water insoluble crosslinked dextrin according to the disclosure can be used in the liquid state, in the solid state or in the semi-solid state. For instance, the cold-water insoluble crosslinked dextrin may be mixed with a small amount of water for obtaining a gel. This gel is then mixed, by kneading and/or mixing, with the active ingredient to be loaded, said active ingredient being in a powdery state or dissolved in an appropriate solvent. Alternatively, the loaded cold-water insoluble crosslinked dextrin may be obtained by adding a selected amount of cold-water insoluble crosslinked dextrin with an excess of guest active ingredient dissolved in suitable solvent, and then stirring overnight at room temperature. The loaded cold-water insoluble crosslinked dextrin can be recovered by filtration under vacuum.

The loaded cold-water insoluble crosslinked dextrin can then be blended with the compound selected from 1-decanoyl-rac-glycerol and CMC.

Use of a Combination of Cold-Water Insoluble Crosslinked Dextrin and a Compound Selected from 1-Decanoyl-Rac-Glycerol and CMC

The invention also relates to the use of a combination:
of a cold-water insoluble crosslinked dextrin,
and of a compound selected from 1-decanoyl-rac-glycerol and CMC,
   for increasing the epithelial permeation of an active ingredient, preferably for increasing the intestinal permeation of an active ingredient, and/or for the epithelial delivery of an active ingredient, preferably for the oral or intrarectal delivery of an active ingredient, preferably for the oral delivery of an active ingredient.

The invention also relates to a method for increasing epithelial permeation of an active ingredient and/or to a method for the epithelial delivery of an active ingredient, comprising administering a combination of a cold-water insoluble crosslinked dextrin and of a compound selected from 1-decanoyl-rac-glycerol and CMC.

Preferably, said cold-water insoluble crosslinked dextrin is as described before. Preferably, said active ingredient is as described before. It is preferably a biopharmaceutical, still preferably a protein, still preferably insulin. Preferably, the amounts and/or ratios of cold-water insoluble crosslinked dextrin and/or of compound selected from 1-decanoyl-rac-glycerol and CMC of said combination are as described before for the composition according to the disclosure.

The term “increasing epithelial permeation” classically means that the combination can increase the passage of an active ingredient from the apical side of the epithelium to the basolateral side of the epithelium. In other words, it means that the combination can increase the crossing of an active ingredient through the epithelium. The term “increasing intestinal permeation” classically means that the combination is able to increase the passage of an active ingredient from the intestinal lumen to the blood compartment. This ability to increase the intestinal permeability can be evaluated by comparing the permeability of said active ingredient to the permeability of said active ingredient in the presence of the alleged permeation enhancer. It may be determined for example by way of a Caco-2 cell permeability assay, in which the active ingredient absorption is measured. It may be determined according to the detailed protocols given herein after in the Examples section.

Preferably, the combination does not impair the cell barrier integrity. This integrity of the cell barrier may be evaluated by the person skilled in the art, by a Caco-2 cell permeability assay in which the lucifer yellow absorption is measured, after exposure to said combination to be tested. It may be evaluated according to the protocol given herein after in the Examples section (“lucifer yellow test”). According to this test, the percentage of lucifer yellow absorbed is equal to or lower than 2.0% dry weight, still preferably equal to or lower than 1.5%, still preferably equal to or lower than 0.7%. This integrity of the membrane may also be evaluated by the person skilled in the art by the well-known TEER measurement, for example according to the protocol given herein after in the Examples section (“TEER measurement”).

The combination may be used by administrating the cold-water insoluble crosslinked dextrin, the compound selected from 1-decanoyl-rac-glycerol and CMC and the active ingredient independently or as composition(s). Preferably, the active ingredient is loaded in the cold-water insoluble crosslinked dextrin. The method/use thus advantageously includes a step of loading (or “associating”) said active ingredient in said cold-water insoluble crosslinked dextrin.

Preferably, the combination is used in the form of a composition according to the disclosure, preferably as described before. It is preferably a composition comprising an active ingredient as described before.

Therefore, preferably, the invention relates to the use of a composition comprising:
a cold-water insoluble crosslinked dextrin; and,
a compound selected from 1-decanoyl-rac-glycerol and CMC; and,
an active ingredient;
   said cold-water insoluble crosslinked dextrin being loaded with said active ingredient, for increasing the epithelial permeation of said active ingredient (preferably for increasing the intestinal permeation of said active ingredient), and/or for the epithelial delivery of said active ingredient (preferably for the oral or intrarectal delivery of said active ingredient, more preferably for the oral delivery of said active ingredient).

Therefore, preferably, the invention also relates to a method for increasing epithelial permeation of an active ingredient and/or to a method for the epithelial delivery of an active ingredient, comprising administering to an organism in need thereof a composition comprising:
a cold-water insoluble crosslinked dextrin; and,
a compound selected from 1-decanoyl-rac-glycerol and CMC; and,
said active ingredient;
   said cold-water insoluble crosslinked dextrin being loaded with said active ingredient.

In the instant disclosure, the amounts of ingredients may be expressed in percentages by weight. Unless otherwise specified these weights are amounts of ingredients as such, in their powdery or oily form. Powdery ingredients generally include small amount of water (also referred to as % moisture or as “loss on drying”) and/or small amounts of impurities. By opposition, in the instant disclosure, when it is referred to dry weight, this well refers to anhydrous weights.

Other characteristics and advantages of the present invention will emerge clearly on reading the examples given hereinafter, which illustrate the invention without however limiting it.

### EXAMPLES

1. Evaluation of the Intestinal Permeation of an Active Ingredient Using a Cold-Water Insoluble Crosslinked Dextrin (Nanosponges) in Combination with Various Permeation Enhancer.

The inventor(s) performed a screening of various permeation enhancers, in combination with nanoparticles of a cold-water insoluble crosslinked dextrin (herein after referred to as “nanosponges”). As the active ingredient, insulin was selected.

### 1.1. Tested Materials

The permeability of insulin was assayed for different samples, referred to as follows (Table 1):

### [Table 1]

| Sample | Reference |
|---|---|
| Insulin-loaded nanosponges | Insulin-loaded nanosponges |
| Insulin alone | Free insulin |
| insulin-loaded nanosponges and different | Insulin-loaded nanosponges + |
| concentrations of permeability enhancer | permeability enhancer - |
| | concentration |

The nanosponges consisted in a cold-water insoluble pea maltodextrin having a DE of 17 and a Mw of 12 000 Da (pea maltodextrin KLEPTOSE® Linecaps, ROQUETTE) crosslinked with sodium trimetaphosphate, obtained according to patent application WO 2021/254662 A1, Example 2 (p. 13 line 8 to p. 14 line 2), the latter being incorporated herein by reference.

The permeation enhancers tested were: 1-decanoyle-rac-glycerol, carboxymethyl cellulose (CMC) salcaprozate sodium (SNAC), lauroyl-L-carnitine, nonaethylene glycol monododecyl ether, polyethylene glycol 3000 50% (w/v) solution, SiO₂ silica 20 μm, SiO₂ silica 150 nm, kaolinite, montmorillonite.

The insulin was bovine insulin (Sigma/15500) for all experiments.

A nanosponges suspension was prepared as follow:
Preparation of a suspension starting from coarse powder of cold-water insoluble crosslinked dextrin in distilled water at the concentration of 10 mg/mL under stirring at room temperature.
Dispersion of the suspension using a high shear homogenizer (Ultraturrax®, IKA, Konigswinter, Germany) for 10 minutes at 24000 rpm.
Use of high-pressure homogenization for 90 minutes at a back-pressure of 500 bar, using an EmulsiFlex C5 instrument (Avastin, USA) for further size reduction.
Purification of homogenized nanosuspension by dialysis (Spectrapore, cellulose membrane, cutoff 12000 Da) to remove synthesis residues potentially present.
Storage of the nanosuspensions at 4 °C.

Insulin-loaded nanosponges were prepared as follows: insulin powder was used to prepare a 2 mg/mL solution in distilled water pH 2.3 adjusted using phosphoric acid. Insulin solution was added to the nanosponges suspension prepared as described above, in a weight ratio insulin solution:nanosponges suspension of 1:5. The mixture was stirred at room temperature for 30 minutes and then centrifuged. The supernatant was separated from the sediment which was collected and freeze-dried. The loading capacity of the nanosponges was of 14±1%, said percentage being expressed in dry weight of insulin with respect to the total dry weight of the nanosponges.

### 1.2. Intestinal Permeation Assay (Caco-2 Assay)

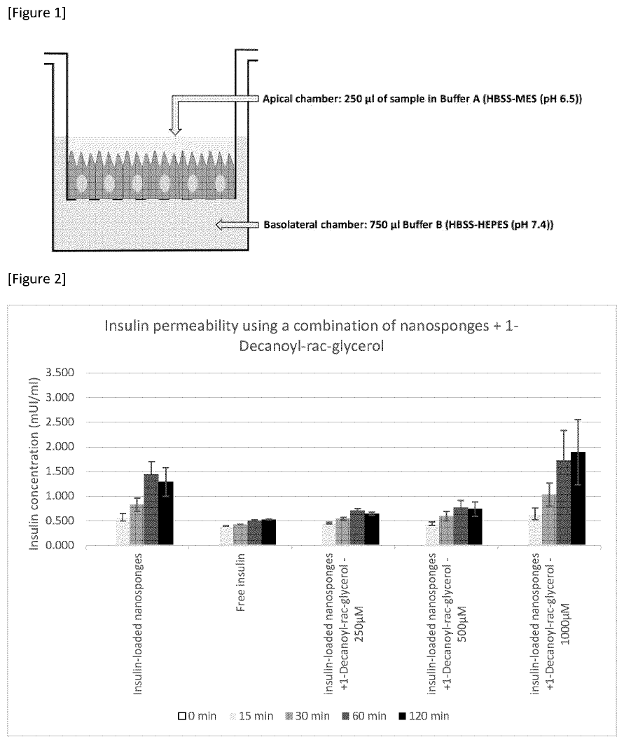

The permeation of insulin was evaluated by way of a Caco-2 assay, using a 24-well Readycell Caco-2 plate (CacoReady 24 Transwell (Costar)-KRECECCR01). Briefly (FIG. 1), samples were prepared at the desired concentrations in Caco-2 buffer A (Hanks' Balanced Salt solution (HBSS)+5 mM 2-(N-morpholino) ethanesulfonic acid (MES) pH 6.5), and 250 μL of each sample were placed in the apical chambers of the Caco-2 plate. To the basolateral chambers, 750 μL of buffer B HBSS-HEPES (4-(2-hydroxyethyl)-1-piperazineethanesulfonic acid) (pH 7.4) was added. The Caco-2 plate was incubated at 37 °C, 5% CO₂, and the permeability of insulin was evaluated by measuring the amount of insulin reaching the basolateral chambers. All materials were tested at non-cytotoxic concentrations, and all samples had a concentration of insulin of 7.5 Ul/mL.

The detailed protocol was as follows.

First, the following stock solutions were prepared:
Free insulin stock solution: insulin was solubilized at 2 mg dry weight/mL in H₂O/HCl PH 2.
Insulin-loaded nanosponges stock solution (prepared extemporaneously): 15 mg of freeze-dried insulin-loaded nanosponges (corresponding to 2.1 mg dry weight of insulin) was added into 1 mL of autoclaved saline solution (water+0.9% NaCl), and gently dispersed until the suspension was homogeneous from a visual inspection. The stock solution had thus 2.1 mg dry weight/mL of insulin, corresponding to 60 Ul/mL of insulin.
permeation enhancers stock solutions: each permeation enhancer was prepared in HBSS buffer (10-fold concentrated as compared to the highest concentration tested).

Then, samples were prepared from those stock solutions into buffer A, so as to obtain the following concentrations (weights are expressed in dry weights):
“Free insulin”: 7.5 Ul/mL of insulin;
“Insulin-loaded nanosponges”: 7.5 Ul/mL of insulin;
“Insulin-loaded nanosponges+1-Decanoyl-rac-glycerol”: 7.5 Ul/mL of insulin+1000 UM or 500 UM or 250 UM of 1-Decanoyl-rac-glycerol (Sigma/M2140).
“Insulin-loaded nanosponges+Sodium carboxymethyl cellulose”: 7.5 Ul/mL of insulin+0.4% or 0.2% or 0.1% (w/v) of CMC (Sigma/419273).
“Insulin-loaded nanosponges+Salcaprozate sodium”: 7.5 Ul/mL of insulin+1 mg/mL or 0.5 mg/mL or 0.25 mg/ml of Salcaprozate sodium (SNAC) 203787-91-1;
“Insulin-loaded nanosponges+Lauroyl-L-carnitine”: 7.5 Ul/mL of insulin+0.25 mM or 0.125 mM or 0.625 mM of Lauroyl-L-carnitine (Sigma/39953);
“Insulin-loaded nanosponges+Nonaethylene glycol monododecyl ether”: 7.5 Ul/mL of insulin+0.01 mM or 0.05 mM or 0.025 mM of Nonaethylene glycol monododecyl ether (Sigma/P9641);
“Insulin-loaded nanosponges+Polyethylene glycol”: 7.5 Ul/mL of insulin+0.2% or 0.1% or 0.05% (w/v) of Polyethylene glycol 3000 (Sigma/81269);
“Insulin-loaded nanosponges+SiO₂ silica 20 μm”: 7.5 Ul/mL of insulin+0.8% or 0.4% or 0.2% (w/v) of SiO₂ silica 20 μm (Sigma/904376);
“Insulin-loaded nanosponges+SiO₂ silica 150 nm”: 7.5 Ul/mL of insulin+0.8% or 0.4% or 0.2% (w/v) of SiO₂ silica 150 nm (Sigma/904414);
“Insulin-loaded nanosponges+Kaolinite”: 7.5 Ul/mL of insulin+0.2% or 0.1% or 0.05% (w/v) of Kaolinite (Sigma/03584);
“Insulin-loaded nanosponges+Montmorillonite”: 7.5 Ul/mL of insulin+0.4% or 0.2% or 0.1% (w/v) of Montmorillonite (Sigma/69866).
   For the “Insulin-loaded nanosponges+permeability enhancer” samples, the permeation enhancer stock solution was first diluted into buffer A, and then the insulin-loaded nanosponges stock solution was added at the target concentration and gently dispersed.

Caco-2 growth media was removed from the 24 well Readycell Caco-2 plate. Then, 250 μL of each sample prepared in buffer A [HBSS-MES (pH 6.5)] were added in the apical chambers. 800 μL of buffer B [HBSS-HEPES (pH 7.4)] were added in the basolateral chambers. 50 μl were collected and transferred into a Greiner 651201 plate for analysis (blank=0 min incubation time=t0). The assays were all performed in triplicate (n=3).

Apical chambers were placed into the basolateral chambers and incubated for 120 minutes (37 °C, 5% CO₂).

At t0+15 min, t0+30 min, t0+60 min, and t0+120 min, 50 μL of medium were collected in the basolateral chamber and transferred into a Greiner plate 651201 for analysis. The detection of insulin was performed using Ultra High Performance Liquid Chromatography coupled to triple-quadrupole Mass Spectrometry (UHPLC-QqQ) method with limits of detection optimized for testing in all permeation enhancers. 50 μL of fresh Buffer B were added to the basolateral chambers. Apical chambers were put back into the basolateral chamber.

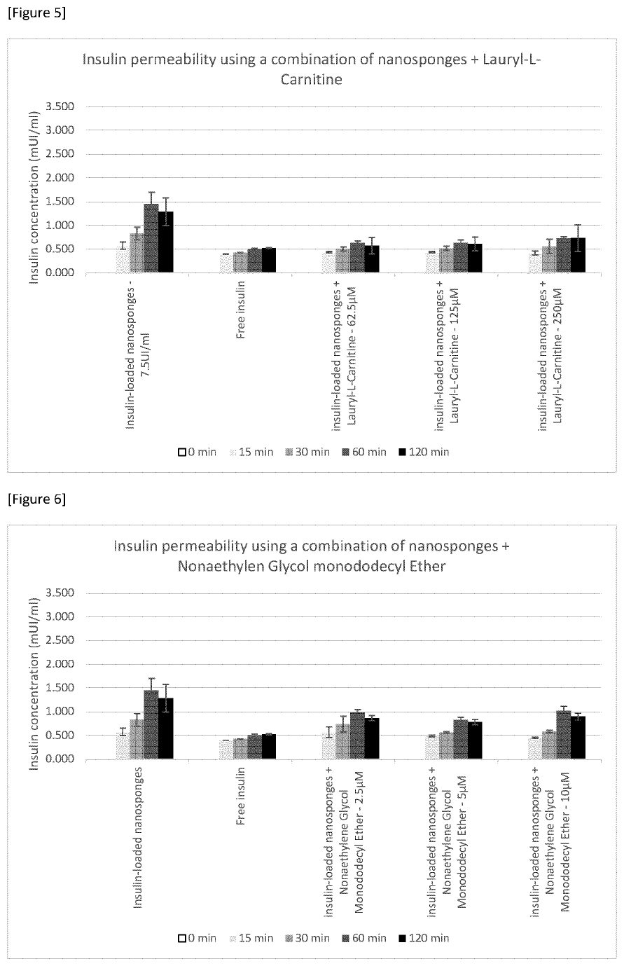

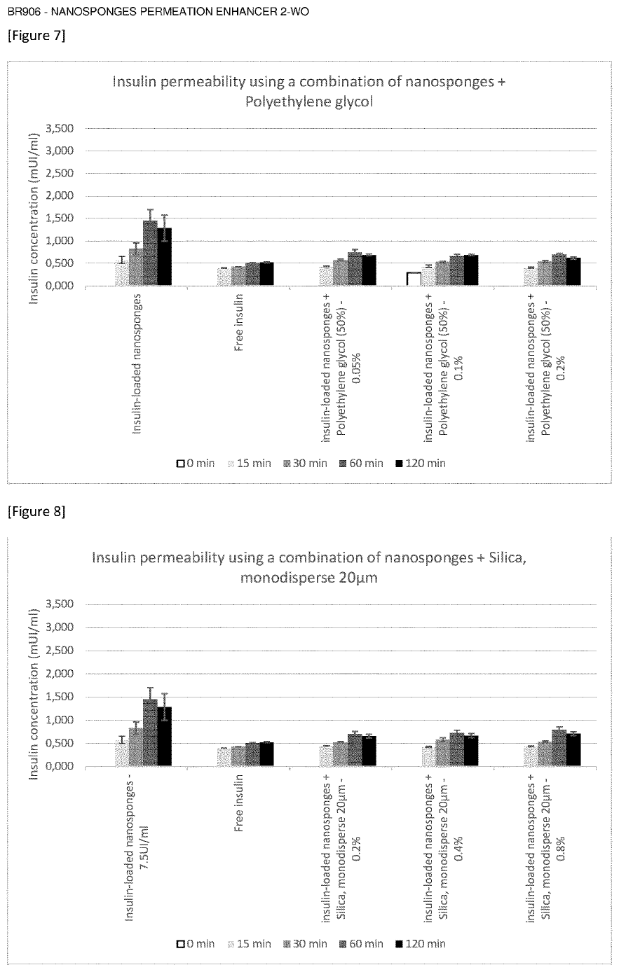

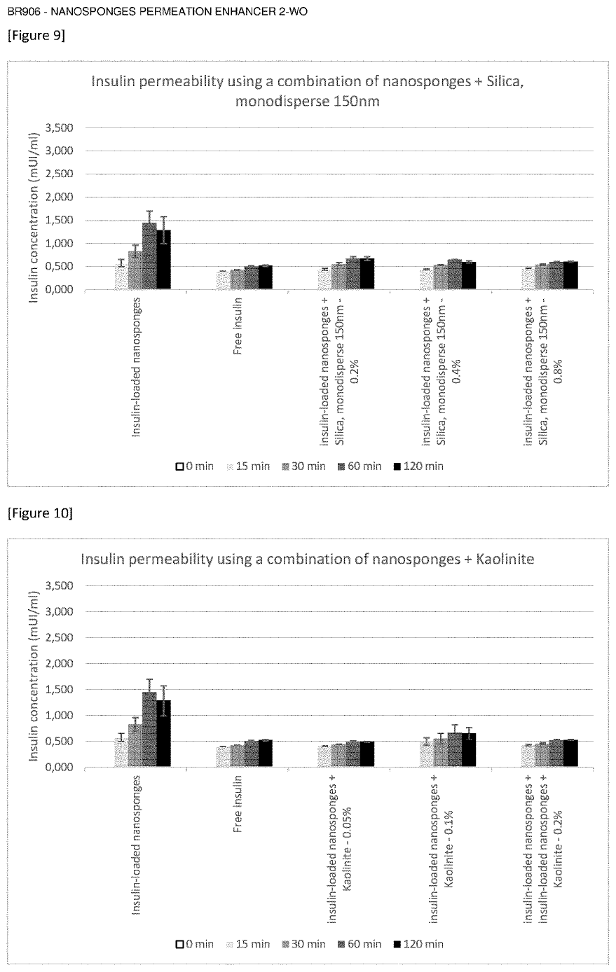

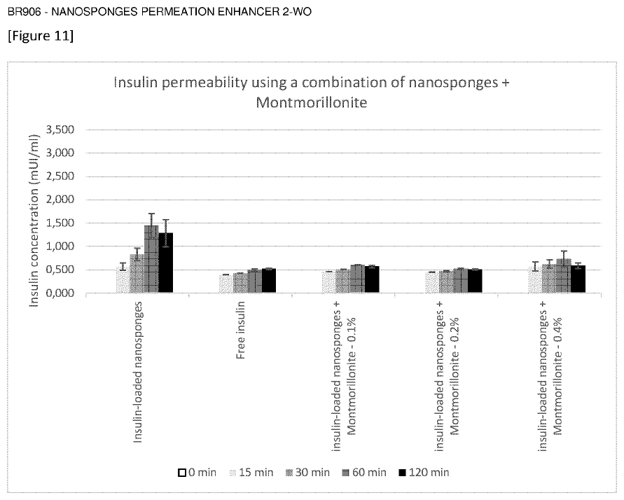

Results are presented in FIGS. 2 to 11.

### 1.3. Results

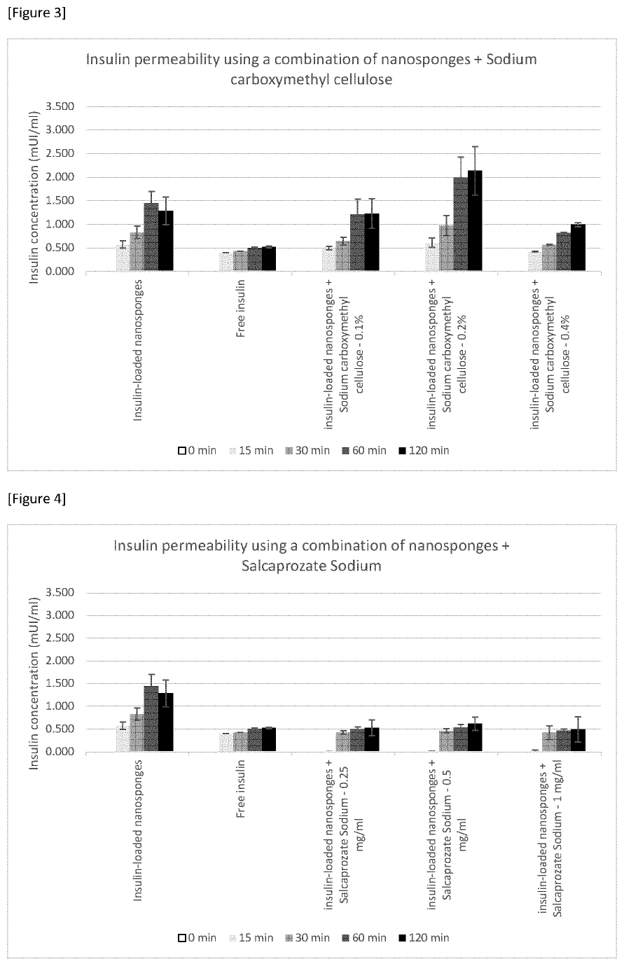

By comparing both controls (“free insulin” versus “insulin-loaded nanosponges”), it can be seen that nanosponges increased the permeation of insulin. Unexpectedly high permeation enhancement was obtained when the nanosponges were combined with 1-Decanoyl-rac-glycerol or CMC (FIGS. 2 and 3). This enhancement was much greater than when the nanosponges alone were used. Although this increase was only observed for the concentrations of 1000 UM and 0.2% for 1-Decanoyl-rac-glycerol and CMC respectively, it should be noted that it doesn't mean that the other concentrations are not performing well. Indeed, the insulin detection method used in this test may not be sensitive enough to detect an increase in all tested concentrations. Regarding the other permeation enhancers, when they were used in combination with the nanosponges, no permeation enhancement was observed (FIGS. 4-11). The level of permeation was like the one obtained with free insulin, and lower than the one obtained with the nanosponges alone. In other words, these permeation enhancers inhibited the permeation effect of the nanosponges.

1. A composition comprising:
a cold-water insoluble crosslinked dextrin; and,
a compound selected from 1-decanoyl-rac-glycerol and carboxymethyl cellulose.

2. The composition of claim 1, wherein said dextrin is selected from pyrodextrins, maltodextrins, cyclodextrins, or from a mixture thereof.

3. The composition of claim 1, wherein said cold-water insoluble crosslinked dextrin is obtainable by reacting a dextrin with a crosslinker, said crosslinker being selected from trimetaphosphate salts, dicarboxylic acids, dianhydrides, carbonyldiimidazole, diphenylcarbonate, triphosgene, acylic dichlorides, diisocyanates, diepoxides, or from any mixture thereof.

4. The composition of claim 1, wherein said cold-water insoluble crosslinked dextrin is obtainable by reacting a dextrin with a crosslinker, wherein the molar ratio of said crosslinker to the anhydroglucose units of said dextrin is equal to or higher than 0.1:1.

5. The composition of claim 1, wherein said cold-water insoluble crosslinked dextrin is in the form of particles.

6. The composition of claim 1, wherein said composition is intended to be administered by epithelial route.

7. The composition of claim 1, wherein said composition is an oral composition.

8. The composition of claim 1 further comprising an active ingredient.

9. The composition of claim 8, wherein said active ingredient is selected from BCS class III drugs, BCS class IV drugs, biopharmaceuticals, or from any mixture thereof.

10. The composition of claim 8, wherein said active ingredient is selected from proteins.

11. The composition of claim 8, wherein said active ingredient is loaded in said cold-water insoluble crosslinked dextrin.

12. The composition of claim 8, for use as a medicament.

13. Non-pharmaceutical use of a composition of claim 8 in a food, nutraceutical, or cosmetic composition.

14. A method for the preparation of a composition according to claim 1, comprising putting into contact said cold-water insoluble crosslinked dextrin with said compound selected from 1-decanoyl-rac-glycerol and carboxymethyl cellulose.

15. Use of a combination:
of a cold-water insoluble crosslinked dextrin,
and of a compound selected from 1-decanoyl-rac-glycerol and carboxymethyl cellulose, for increasing the epithelial permeation of an active ingredient, preferably for increasing the intestinal permeation of an active ingredient.

16. Use of a combination:
of a cold-water insoluble crosslinked dextrin,
and of a compound selected from 1-decanoyl-rac-glycerol and carboxymethyl cellulose, for the epithelial delivery of an active ingredient, preferably for the oral or intrarectal delivery of an active ingredient, more preferably for the oral delivery of an active ingredient.

17. The use of claim 15, wherein said active ingredient is selected from BCS class III drugs, BCS class IV drugs, biopharmaceuticals, or from any mixture thereof.

18. The use of claim 15, wherein said active ingredient is selected from proteins.
